SEQUENCE LISTING (15 sequences)

SEQ ID NO: 1 - protein, 4 aa, Artificial Sequence
  Cell adhesion peptide
RGDG

SEQ ID NO: 2 - protein, 4 aa, Artificial Sequence
  Cell adhesion peptide
RGDA

SEQ ID NO: 3 - protein, 4 aa, Artificial Sequence
  Cell adhesion peptide
RGDV

SEQ ID NO: 4 - protein, 4 aa, Artificial Sequence
  Cell adhesion peptide
RGDS

SEQ ID NO: 5 - protein, 4 aa, Artificial Sequence
  Cell adhesion peptide
RGDT

SEQ ID NO: 6 - protein, 4 aa, Artificial Sequence
  Cell adhesion peptide
RGDF

SEQ ID NO: 7 - protein, 4 aa, Artificial Sequence
  Cell adhesion peptide
RGDM

SEQ ID NO: 8 - protein, 4 aa, Artificial Sequence
  Cell adhesion peptide
RGDP

SEQ ID NO: 9 - protein, 4 aa, Artificial Sequence
  Cell adhesion peptide
RGDN

SEQ ID NO: 10 - protein, 5 aa, Artificial Sequence
  Cell adhesion peptide
YIGSR

SEQ ID NO: 11 - protein, 5 aa, Artificial Sequence
  Cell adhesion peptide
PDSGR

SEQ ID NO: 12 - protein, 4 aa, Artificial Sequence
  Cell adhesion peptide
REDV

SEQ ID NO: 13 - protein, 5 aa, Artificial Sequence
  Cell adhesion peptide
IDAPS

SEQ ID NO: 14 - protein, 6 aa, Artificial Sequence
  Cell adhesion peptide
KQAGDV

SEQ ID NO: 15 - protein, 5 aa, Artificial Sequence
  Cell adhesion peptide
GRGDS

(19) United States
(12) Patent Application Publication
(10) Pub. No.: US 2022/0145263 A1
(43) Pub. Date: May 12, 2022

(54) CELL CULTURE SCAFFOLD MATERIAL, RESIN FILM, CELL CULTURE VESSEL, AND METHOD FOR CULTURING A CELL

(71) Applicant: SEKISUI CHEMICAL CO., LTD., Osaka (JP)

(72) Inventors: Satoshi HANEDA, Osaka (JP); Yoshiki TOMITA, Osaka (JP)

(73) Assignee: SEKISUI CHEMICAL CO., LTD., Osaka (JP)

(21) Appl. No.: 17/380,214

(22) Filed: Jul. 20, 2021

(30) Foreign Application Priority Data
Nov. 12, 2020 (JP) 2020-188878

(51) Int. Cl. C12N 5/074

(52) U.S. Cl. CPC C12N 5/0696; C12N 2513/00; C12N 2537/10; C12N 2533/50; C12N 2533/30

(57) ABSTRACT

To provide a cell culture scaffold material capable of enhancing adhesiveness of cells. The cell culture scaffold material according to the present invention contains a peptide-conjugated acrylic resin, in which the peptide-conjugated acrylic resin has a first structural part having no peptide portion in a side chain and a second structural part having a peptide portion in a side chain, and solubility parameter calculated by Okitsu's equation for the first structural part is 9.7 (cal/cm³)^{1/2} or more and 10.7 (cal/cm³)^{1/2} or less.

### BACKGROUND OF THE INVENTION

### Field of the Invention

The present invention relates to a cell culture scaffold material. Also, the present invention relates to a resin film using the cell culture scaffold material. Further, the present invention relates to a cell culture vessel and a method for culturing cells using the resin film.

### Description of the Related Art

Cells of animals such as human, mouse, rat, pig, cow and monkey are used in research and development in academic fields, drug discovery fields, regenerative medicine fields, and the like. As a scaffold material used for culturing animal cells, adhesive proteins such as laminin and vitronectin, and natural polymer materials such as matrigel derived from mouse sarcoma are used.

Further, a scaffold material using a synthetic resin and a scaffold material using a synthetic resin to which a peptide is bound are also known.

For example, Patent Document 1 below discloses an article for cell culture coated with a composition containing a polymer in which an acrylic polymer and a polypeptide are bound. In Patent Document 1, as the acrylic polymer, a hydrophilic acrylic polymer obtained by polymerizing a hydrophilic acrylic monomer is used.

### RELATED ART DOCUMENTS

### Patent Documents

Patent Document 1: WO 2012/158235 A2

### SUMMARY OF THE INVENTION

### Problems to be Solved by the Invention

As described in Patent Document 1, a cell culture scaffold material using an acrylic resin to which a peptide is bound is known. However, with the scaffold material described in Patent Document 1, it is difficult to sufficiently enhance adhesiveness of cells.

An object of the present invention is to provide a cell culture scaffold material capable of enhancing adhesiveness of cells. Also, an object of the present invention is to provide a resin film using the cell culture scaffold material. Further, an object of the present invention is to provide a cell culture vessel and a method for culturing cells using the resin film.

According to a broad aspect of the present invention, a cell culture scaffold material containing a peptide-conjugated acrylic resin, in which the peptide-conjugated acrylic resin has a first structural part having no peptide portion in a side chain and a second structural part having a peptide portion in a side chain, and solubility parameter calculated by Okitsu's equation for the first structural part is 9.7 (cal/cm³)^{1/2} or more and 10.7 (cal/cm³)^{1/2} or less is provided.

In a certain aspect of the cell culture scaffold material according to the present invention, the first structural part has a poly(meth)acrylic acid ester skeleton.

In a certain aspect of the cell culture scaffold material according to the present invention, the number of amino acid residues in the peptide portion in the second structural part is 10 or less.

According to a broad aspect of the present invention, a resin film formed of the above-mentioned cell culture scaffold material is provided.

In a certain aspect of the resin film according to the present invention, compressive elastic modulus at a frequency of 1 Hz measured in ion exchange water after immersing the resin film in the ion exchange water at 37° C. for 24 hours in accordance with ISO14577-1 using a nanoindenter device is 1 GPa or more.

In a certain aspect of the resin film according to the present invention, the resin has a water swelling ratio of 70% or less.

In a certain aspect of the resin film according to the present invention, the resin film has a sea-island structure, and the island portion in the sea-island structure contains the peptide portion.

According to a broad aspect of the present invention, a cell culture vessel provided with the above-mentioned resin film in at least a part of cell culture area is provided.

According to a broad aspect of the present invention, a method for culturing cells, including the step of seeding cells on the above-mentioned resin film, is provided.

### Effect of the Invention

The cell culture scaffold material according to the present invention contains a peptide-conjugated acrylic resin, and the peptide-conjugated acrylic resin has a first structural part having no peptide portion in a side chain and a second structural part having a peptide portion in a side chain. In the cell culture scaffold material according to the present invention, solubility parameter calculated by Okitsu's equation for the first structural part is 9.7 (cal/cm³)^{1/2} or more and 10.7 (cal/cm³)^{1/2} or less. The cell culture scaffold material according to the present invention has the above constitution and thus can enhance adhesiveness of cells.

### BRIEF DESCRIPTION OF THE DRAWINGS

The FIGURE is a cross-sectional view schematically showing a cell culture vessel according to an embodiment of the present invention where 1 is the cell culture vessel, 2 is the vessel body, 2a is a surface and 3 is the resin film.

### DETAILED DESCRIPTION OF THE PREFERRED EMBODIMENTS

Hereinafter, the details of the present invention will be described.

The cell culture scaffold material according to the present invention (hereinafter, may be abbreviated as “scaffold material”) contains a peptide-conjugated acrylic resin, and the peptide-conjugated acrylic resin has a first structural part having no peptide portion in a side chain and a second structural part having a peptide portion in a side chain. In the cell culture scaffold material according to the present invention, solubility parameter calculated by Okitsu's equation for the first structural part is 9.7 (cal/cm³)^{1/2} or more and 10.7 (cal/cm³)^{1/2} or less.

The scaffold material according to the present invention has the above constitution and thus can enhance adhesiveness of cells. The scaffold material according to the present invention can maintain high cell adhesiveness for a long period of time.

It is difficult to sufficiently enhance the adhesiveness of cells with a conventional scaffold material using an acrylic resin to which a peptide is bound (peptide-conjugated acrylic resin). The present inventors have found that the adhesiveness of cells cannot be sufficiently enhanced in the conventional scaffold material using the peptide-conjugated acrylic resin because an acrylic resin having high hydrophilicity is used. The present inventors have found that the adhesiveness of cells can be enhanced by setting solubility parameter (SP value) of a specific structural part in a specific range in the acrylic resin to which the peptide is bound to improve balance between hydrophilicity and hydrophobicity of the structural part.

Further, since the conventional scaffold material uses an acrylic resin having high hydrophilicity, the scaffold material swells excessively during cell culture, the scaffold material peels off from a vessel or the like, or the adhesiveness of cells varies by lot, and it is difficult to use for mass culture of cells. On the other hand, with respect to the scaffold material according to the present invention, the scaffold material is hard to peel off from the vessel or the like during cell culture, and variation in performance between lots can be suppressed, so that it can also be used for mass culture of cells.

(Peptide-Conjugated Acrylic Resin)

The scaffold material contains a peptide-conjugated acrylic resin. The peptide-conjugated acrylic resin is an acrylic resin to which a peptide is bound. The peptide-conjugated acrylic resin has a main chain and a side chain. The peptide-conjugated acrylic resin has a part having no peptide portion in a side chain (first structural part) and a part having a peptide portion in a side chain (second structural part). As the peptide-conjugated acrylic resin, only one type may be used, or two or more types may be used in combination.

The first structural part is a structural part excluding the second structural part in the peptide-conjugated acrylic resin. For example, when the peptide-conjugated acrylic resin is composed of M structural units (A) having no peptide portion and N structural units (B) having a peptide portion, the first structural part is a part composed of M structural units (A), and the second structural part is a part composed of N structural units (B). The structural unit (A) may be only one type of structural unit, or may be two or more types of structural units. The structural unit (B) may be only one type of structural unit, or may be two or more types of structural units. Further, the structural unit (A) and the structural unit (B) may be present alternately, randomly, or in blocks in the peptide-conjugated acrylic resin, and a part of the structural unit may be grafted and present.

In addition, in this specification, “(meth)acrylic” means one or both of “acrylic” and “methacrylic”, and “(meth)acrylate” means one or both of “acrylate” and “methacrylate”.

<First Structural Part>

The first structural part is a structural part having no peptide portion in a side chain in the peptide-conjugated acrylic resin. The first structural part is preferably a part composed of structural units having no peptide portion in the peptide-conjugated acrylic resin. The first structural part preferably has a structural unit having no peptide portion as a repeating structural unit.

The first structural part preferably has a structural unit derived from a (meth)acrylic acid ester. The first structural part may have only a structural unit derived from a (meth)acrylic acid ester. The first structural part preferably has a poly(meth)acrylic acid ester skeleton.

The first structural part may or may not have a structural unit derived from a monomer other than the (meth)acrylic acid ester.

Types of the (meth)acrylic acid ester and monomer other than the (meth)acrylic acid ester are not particularly limited. In the present invention, types of the (meth)acrylic acid ester and monomer other than the (meth)acrylic acid ester and the like are selected as appropriate so that the solubility parameter (SP value) of the first structural part satisfies a specific range.

Examples of the (meth)acrylic acid ester include (meth)acrylic acid alkyl ester, (meth)acrylic acid cyclic alkyl ester, (meth)acrylic acid aryl ester, polyethylene glycol (meth)acrylates, phosphorylcholine (meth)acrylate, and the like. As the (meth)acrylic acid ester, only one type may be used, or two or more types may be used in combination.

Examples of the (meth)acrylic acid alkyl ester include methyl (meth)acrylate, ethyl (meth)acrylate, n-propyl (meth)acrylate, isopropyl (meth)acrylate, n-butyl (meth)acrylate, isobutyl (meth)acrylate, t-butyl (meth)acrylate, n-octyl (meth)acrylate, isooctyl (meth)acrylate, 2-ethylhexyl (meth)acrylate, nonyl (meth)acrylate, isononyl (meth)acrylate, decyl (meth)acrylate, isodecyl (meth)acrylate, lauryl (meth)acrylate, stearyl (meth)acrylate, isotetradecyl (meth)acrylate, and the like.

The (meth)acrylic acid alkyl ester may be substituted with a substituent such as an alkoxy group having 1 to 3 carbon atoms and a tetrahydrofurfuryl group. Examples of such (meth)acrylic acid alkyl ester include methoxyethyl acrylate, tetrahydrofurfuryl acrylate, and the like.

Examples of the (meth)acrylic acid cyclic alkyl ester include cyclohexyl (meth)acrylate, isobornyl (meth)acrylate, and the like.

Examples of the (meth)acrylic acid aryl ester include phenyl (meth)acrylate, benzyl (meth)acrylate, and the like.

Examples of the polyethylene glycol (meth)acrylates include methoxy-polyethylene glycol (meth)acrylate, ethoxy-polyethylene glycol (meth)acrylate, hydroxy-polyethylene glycol (meth)acrylate, methoxy-diethylene glycol (meth)acrylate, ethoxy-diethylene glycol (meth)acrylate, hydroxy-diethylene glycol (meth)acrylate, methoxy-triethylene glycol (meth)acrylate, ethoxy-triethylene glycol (meth)acrylate, hydroxy-triethylene glycol (meth)acrylate, and the like.

Examples of the phosphorylcholine (meth)acrylate include 2-(meth)acryloyloxyethyl phosphorylcholine and the like.

Examples of the monomer other than the (meth)acrylic acid ester include (meth)acrylamides, vinyl compounds, and the like.

As the monomer other than the (meth)acrylic acid ester, only one type may be used, or two or more types may be used in combination.

Examples of the (meth)acrylamides include (meth)acrylamide, N-isopropyl (meth)acrylamide, N-tert-butyl (meth)acrylamide, N,N′-dimethyl (meth)acrylamide, (3-(meth)acrylamide propyl)trimethylammonium chloride, 4-(meth)acryloylmorpholine, 3-(meth)acryloyl-2-oxazolidinone, N-[3-(dimethylamino)propyl](meth)acrylamide, N-(2-hydroxyethyl) (meth)acrylamide, N-methylol(meth)acrylamide, 6-(meth)acrylamide hexanoic acid, and the like.

Examples of the vinyl compound include ethylene, allylamine, vinylpyrrolidone, maleic anhydride, maleimide, itaconic acid, (meth)acrylic acid, vinylamine, and the like.

From the viewpoint of easily adjusting the solubility parameter (SP value), the first structural part preferably has a structural unit derived from two or more types of (meth)acrylic acid esters.

The solubility parameter (SP value) calculated by Okitsu's equation for the first structural part is 9.7 (cal/cm³)^{1/2} or more and 10.7 (cal/cm³)^{1/2} or less. When the solubility parameter (SP value) is less than 9.7 (cal/cm³)^{1/2} or exceeds 10.7 (cal/cm³)^{1/2} it is difficult to enhance adhesiveness of cells, and it is particularly difficult to enhance adhesiveness of cells for a long period of time. Also, when the solubility parameter (SP value) exceeds 10.7 (cal/cm³)^{1/2}, the scaffold material may peel off from the vessel or the like during cell culture.

The solubility parameter (SP value) calculated by Okitsu's equation for the first structural part is preferably 9.8 (cal/cm³)^{1/2} or more, more preferably 10.0 (cal/cm³)^{1/2} or more, and further preferably 10.2 (cal/cm³)^{1/2} or more. The solubility parameter (SP value) calculated by Okitsu's equation for the first structural part is preferably 10.5 (cal/cm³)^{1/2} or less, more preferably 10.3 (cal/cm³)^{1/2} or less, and further preferably 10.2 (cal/cm³)^{1/2} or less. When the solubility parameter (SP value) is the above lower limit or more and the above upper limit or less, the adhesiveness of cells can be even more enhanced. Further, when the solubility parameter (SP value) is the above upper limit or less, it is possible to effectively suppress peeling of the scaffold material from the vessel or the like during cell culture.

More specifically, the solubility parameter (SP value) is calculated from a constant of ΔF of Okitsu (Toshinao Okitsu, “Adhesion”, Vol. 40, No. 8, p. 342 (1996)).

The average ratio of the structural units derived from the (meth)acrylic acid ester to 100 mol % of the total structural units of the first structural part is preferably 20 mol % or more, more preferably 30 mol % or more, even more preferably 40 mol % or more, further preferably 50 mol % or more, further even more preferably 60 mol % or more, and particularly preferably 70 mol % or more. When the average ratio of the structural units derived from the (meth)acrylic acid ester is the above lower limit or more, the solubility parameter (SP value) can be easily adjusted, and the effect of the present invention can be even more effectively exhibited. The average ratio of the structural units derived from the (meth)acrylic acid ester to 100 mol % of the total structural units of the first structural part may be 80 mol % or more, 90 mol % or more, 95 mol % or more, or 100 mol %. The average ratio of the structural units derived from the (meth)acrylic acid ester to 100 mol % of the total structural units of the first structural part may be 100 mol % or less, or 90 mol % or less.

The content of the first structural part in 100% by weight of the peptide-conjugated acrylic resin is preferably 20% by weight or more, more preferably 30% by weight or more, further preferably 40% by weight or more, preferably 95% by weight or less, more preferably 90% by weight or less, and further preferably 85% by weight or less. When the content of the first structural part is the above lower limit or more and the above upper limit or less, the effect of the present invention can be even more effectively exhibited.

The content of the structural unit derived from the (meth)acrylic acid ester in 100% by weight of the peptide-conjugated acrylic resin is preferably 20% by weight or more, more preferably 30% by weight or more, further preferably 40% by weight or more, preferably 95% by weight or less, more preferably 90% by weight or less, and further preferably 85% by weight or less. When the content of the structural unit derived from the (meth)acrylic acid ester is the above lower limit or more and the above upper limit or less, the effect of the present invention can be even more effectively exhibited.

<Second Structural Part>

The second structural part is a structural part of the peptide-conjugated acrylic resin having a peptide portion in a side chain. The second structural part is a part composed of structural units having a peptide portion in the peptide-conjugated acrylic resin. The second structural part preferably has a structural unit having a peptide portion as a repeating structural unit.

The second structural part preferably has a structural unit having a skeleton derived from a peptide and a skeleton derived from a compound having a functional group capable of binding to the peptide. The structural unit having a peptide portion preferably has a skeleton derived from a peptide and a skeleton derived from a compound having a functional group capable of binding to the peptide.

In the present specification, the above-mentioned “compound having a functional group capable of binding to the peptide” may be described as “compound X”.

Therefore, the second structural part preferably has a structural unit having a skeleton derived from a peptide and a skeleton derived from compound X. The structural unit having a peptide portion preferably has a skeleton derived from a peptide and a skeleton derived from compound X. In a structural unit having a skeleton derived from a peptide and a skeleton derived from compound X, the peptide and compound X are bound to each other. As the peptide and the compound X, only one type may be used, or two or more types may be used in combination, respectively.

The functional group capable of binding to the peptide is preferably a functional group capable of condensing with the carboxyl group or amino group of the peptide.

Examples of the functional group capable of binding to the peptide include a carboxyl group, a thiol group, an amino group, a hydroxyl group, a cyano group, and the like.

The compound X preferably has a functional group capable of binding to a (meth)acrylic acid ester.

Examples of the functional group capable of binding to the (meth)acrylic acid ester include a vinyl group, a (meth)acryloyl group, an allyl group, and the like.

The compound X preferably has a carboxyl group or an amino group as the functional group capable of binding to the peptide. The compound X more preferably has a (meth)acryloyl group as a functional group capable of binding to the (meth)acrylic acid ester.

The compound X is preferably a compound having a carboxyl group or an amino group and having a (meth)acryloyl group.

Examples of the compound X include (meth)acrylic acid, itaconic acid, acrylamide, and the like. As the compound X, only one type may be used, or two or more types may be used in combination.

The compound X is preferably (meth)acrylic acid or itaconic acid, and more preferably (meth)acrylic acid.

The number of amino acid residues of the peptide portion in the second structural part is preferably 3 or more, more preferably 4 or more, further preferably 5 or more, preferably 10 or less, more preferably 8 or less, and further preferably 6 or less. When the number of amino acid residues is the above lower limit or more and the above upper limit or less, the adhesiveness to the seeded cells can be even more enhanced, and cell proliferation rate can be even more enhanced. However, the number of amino acid residues of the peptide portion may exceed 10 or 15.

The peptide portion preferably has a cell-adhesive amino acid sequence. The cell-adhesive amino acid sequence refers to an amino acid sequence whose cell adhesion activity has been confirmed by phage display method, sepharose beads method, or plate coating method. As the phage display method, for example, a method described in “The Journal of Cell Biology, Volume 130, Number 5, September 1995 1189-1196” can be used. As the sepharose beads method, for example, a method described in “Protein, Nucleic Acid and Enzyme, Vol. 45 No. 15 (2000) 2477” can be used. As the plate coating method, for example, a method described in “Protein, Nucleic Acid and Enzyme, Vol. 45 No. 15 (2000) 2477” can be used.

Examples of the cell-adhesive amino acid sequence include RGD sequence (Arg-Gly-Asp), YIGSR sequence (Tyr-Ile-Gly-Ser-Arg), PDSGR sequence (Pro-Asp-Ser-Gly-Arg), HAV sequence (His-Ala-Val), ADT sequence (Ala-Asp-Thr), QAV sequence (Gln-Ala-Val), LDV sequence (Leu-Asp-Val), IDS sequence (Ile-Asp-Ser), REDV sequence (Arg-Glu-Asp-Val), IDAPS sequence (Ile-Asp-Ala-Pro-Ser), KQAGDV sequence (Lys-Gln-Ala-Gly-Asp-Val), TDE sequence (Thr-Asp-Glu), and the like. In addition, examples of the cell-adhesive amino acid sequence include sequences described in “Medicina Philosophica, Vol. 9, No. 7, pp. 527-535, 1990” and “Journal of Osaka Women's and Children's Hospital, Vol. 8, No. 1, pp. 58-66, 1992”, and the like. The peptide portion may have only one type of cell-adhesive amino acid sequence, or may have two or more types.

The cell-adhesive amino acid sequence preferably has at least one of the above-mentioned cell-adhesive amino acid sequences, more preferably has at least an RGD sequence, a YIGSR sequence or a PDSGR sequence, and further preferably has at least an RGD sequence represented by the following formula (1) In this case, the adhesiveness to the seeded cells can be even more enhanced, and the cell proliferation rate can be even more enhanced.

Arg-Gly-Asp-X Formula (1)

In the above formula (1), X represents Gly, Ala, Val, Ser, Thr, Phe, Met, Pro, or Asn.

The peptide portion may be linear or may have a cyclic peptide skeleton. The cyclic peptide skeleton is a cyclic skeleton composed of a plurality of amino acids. From the viewpoint of more effectively exhibiting the effect of the present invention, the cyclic peptide skeleton is preferably composed of 4 or more amino acids, preferably composed of 5 or more amino acids, and preferably composed of 10 or less amino acids.

In the peptide-conjugated acrylic resin, the content of the peptide portion is preferably 0.1 mol % or more, more preferably 1 mol % or more, further preferably 5 mol % or more, and particularly preferably 10 mol % or more. In the peptide-conjugated acrylic resin, the content of the peptide portion is preferably 60 mol % or less, more preferably 50 mol % or less, further preferably 35 mol % or less, and particularly preferably 25 mol % or less. When the content of the peptide portion is the above lower limit or more, a phase-separated structure can be even more easily formed. When the content of the peptide portion is the above lower limit or more, the adhesiveness to the seeded cells can be even more enhanced, and the cell proliferation rate can be even more enhanced. Further, when the content of the peptide portion is the above upper limit or less, production cost can be suppressed. The content (mol %) of the peptide portion is the amount of substance of the peptide portion with respect to the total of the amount of substance of structural units constituting a synthetic resin having the peptide portion.

The content of the peptide portion can be measured by FT-IR or LC-MS.

The content of the second structural part in 100 mol % of the peptide-conjugated acrylic resin is preferably 0.1 mol % or more, more preferably 0.5 mol % or more, further preferably 1 mol % or more, preferably 60 mol % or less, more preferably 50 mol % or less, and further preferably 35 mol % or less. When the content of the second structural part is the above lower limit or more and the above upper limit or less, the effect of the present invention can be even more effectively exhibited.

<Other Details of Peptide-Conjugated Acrylic Resin>

The weight average molecular weight of the peptide-conjugated acrylic resin is preferably 50,000 or more, more preferably 100,000 or more, preferably 1,000,000 or less, and more preferably 800,000 or less. When the weight average molecular weight is the above lower limit or more and the above upper limit or less, the effect of the present invention can be even more effectively exhibited. When the weight average molecular weight is the above upper limit or less, extensibility of cells in cell culture can be even more effectively exhibited.

The weight average molecular weight of the peptide-conjugated acrylic resin can be measured by, for example, the following method. The peptide-conjugated acrylic resin is dissolved in tetrahydrofuran (THF) to prepare a 0.2% solution of the peptide-conjugated acrylic resin. Next, evaluation is performed using a gel permeation chromatography (GPC) measuring device (APC system, manufactured by Waters Corporation) under the following measurement conditions.
Column: HSPgel HR MB-M 6.0×150 mm
Flow rate: 0.5 mL/min
Column temperature: 40° C.
Injection volume: 10 μl
Detector: RI, PDA
Standard sample: Polystyrene

(Cell Culture Scaffold Material)

The scaffold material is used for culturing cells. The scaffold material is used as a scaffold for cells when culturing the cells.

Examples of the cells include cells of animals such as human, mouse, rat, pig, cow and monkey. In addition, examples of the cells include somatic cells and the like, and examples thereof include stem cells, progenitor cells, mature cells, and the like. The somatic cells may be cancer cells.

Examples of the stem cells include mesenchymal stem cells (MSCs), iPS cells, ES cells, Muse cells, embryonic cancer cells, embryonic germ stem cells, mGS cells, and the like.

Examples of the mature cells include nerve cells, cardiomyocytes, retinal cells, hepatocytes, and the like.

The scaffold material is preferably used for two-dimensional culture (plane culture), three-dimensional culture or suspension culture of cells, and more preferably used for two-dimensional culture (plane culture).

The scaffold material is preferably used for serum-free medium culture. Since the scaffold material contains the peptide-conjugated acrylic resin, the adhesiveness of cells can be enhanced even in a serum-free medium culture containing no feeder cell or adhesive protein, and in particular, initial fixation rate after cell seeding can be even more enhanced.

The content of the peptide-conjugated acrylic resin in 100% by weight of the scaffold material is preferably 90% by weight or more, more preferably 95% by weight or more, further preferably 97.5% by weight or more, particularly preferably 99% by weight or more, and most preferably 100% by weight (total amount). Therefore, it is most preferable that the scaffold material contains only the peptide-conjugated acrylic resin. When the content of the peptide-conjugated acrylic resin is the above lower limit or more, the effect of the present invention can be even more effectively exhibited.

The scaffold material may contain components other than the peptide-conjugated acrylic resin. Ingredients other than the peptide-conjugated acrylic resin include polyolefin resins, polyether resins, polyvinyl alcohol resins, polyesters, epoxy resins, polyamide resins, polyimide resins, polyurethane resins, polycarbonate resins, polysaccharides, celluloses, polypeptides, synthetic peptides, and the like.

From the viewpoint of effectively exhibiting the effect of the present invention, the smaller the content of the components other than the peptide-conjugated acrylic resin, the better. The content of the component in 100% by weight of the scaffold material is preferably 10% by weight or less, more preferably 5% by weight or less, further preferably 2.5% by weight or less, particularly preferably 1% by weight or less, and most preferably 0% by weight (not contained). Therefore, it is most preferable that the scaffold material contains no component other than the peptide-conjugated acrylic resin.

It is preferable that the scaffold material does not substantially contain animal-derived raw materials. Since it does not contain animal-derived raw materials, it is possible to provide a cell culture scaffold material that is highly safe and has little variation in quality during production. In addition, the phrase “does not substantially contain animal-derived raw materials” means that the animal-derived raw materials in the scaffold material are 3% by weight or less. In the above scaffold material, the animal-derived raw materials in the scaffold material is preferably 1% by weight or less, and most preferably 0% by weight. That is, it is most preferable that the scaffold material does not contain any animal-derived raw materials.

The shape of the scaffold material is not particularly limited. The scaffold material may be particles, fibers, porous body, or film. The particles, the fibers, the porous body, and the film may each contain components other than the scaffold material.

In addition, the scaffold material can also be used as a carrier (medium) for cell culture containing the scaffold material and polysaccharides. The polysaccharide is not particularly limited, and a conventionally known polysaccharide can be used. The polysaccharide is preferably a water-soluble polysaccharide.

Further, the scaffold material can also be used as a fiber for cell culture having a fiber body and a scaffold material arranged on the surface of the fiber body. In this case, the cell culture scaffold material is preferably coated on the surface of the fiber body, and is preferably a coated material. In this fiber for cell culture, a scaffold material may be present in the fiber body. For example, the scaffold material can be present in the fiber body by impregnating or kneading the fiber body into the liquid scaffold material. In general, stem cells have a property of being difficult to adhere to a planar structure and easily adhering to a three-dimensional structure such as a fibrous structure. Therefore, a fiber for cell culture is suitably used for three-dimensional culture of stem cells. Among stem cells, it is more preferably used for three-dimensional culture of adipose stem cells.

<Resin Film>

The resin film according to the present invention is a resin film formed of the above-mentioned scaffold material. The resin film is formed by using a cell culture scaffold material. The resin film is preferably a film-like scaffold material. The resin film is preferably a film-like material for scaffold material.

Thickness of the resin film is not particularly limited. The resin film may have an average thickness of 50 nm or more, 500 nm or more, 1000 μm or less, or 500 μm or less.

Compressive elastic modulus at a frequency of 1 Hz measured in ion exchange water after immersing the resin film in the ion exchange water at 37° C. for 24 hours in accordance with ISO14577-1 using a nanoindenter device is preferably 1 GPa or more, more preferably 1.5 GPa or more, and further preferably 2 GPa or more. When the compressive elastic modulus is the above lower limit or more, extensibility of cells in cell culture can be enhanced. The upper limit of the compressive elastic modulus is not particularly limited. The compressive elastic modulus may be 15 GPa or less.

The compressive elastic modulus is measured as follows.

After putting the resin film in a beaker filled with ion exchange water, the beaker is left in a constant temperature bath at 37° C. for 24 hours. The resin film immersed for 24 hours is measured at a frequency of 1 Hz in accordance with ISO14577-1 in ion exchange water using a nanoindenter device (for example, Triboindenter, manufactured by Hysitron Inc.). The compressive elastic modulus is calculated according to the following formula.

Compressive elastic modulus=√π×(Slope of load-displacement curve in elastic region)/(2×√(Contact projected area))

Here, the elastic region refers to a region where the slope of the load-displacement curve is constant. Also, the contact projected area refers to the area where an indenter and the sample come into contact with each other.

Berkovich (triangular pyramid shape, tip diameter R is several hundred nanometers) is used as the indenter, and the indentation depth can be 50 nm.

The compressive elastic modulus can be satisfactorily adjusted, for example, by adjusting the type and weight average molecular weight of the peptide-conjugated acrylic resin. For example, swelling in water is suppressed by reducing the SP value of the peptide-conjugated acrylic resin, increasing the weight average molecular weight, or forming a crosslinked structure between molecules, and the compressive elastic modulus can be increased.

The resin film has a water swelling ratio of preferably 70% or less, more preferably 50% or less, even more preferably 40% or less, further preferably 30% or less, and particularly preferably 20% or less. When the water swelling ratio is the above upper limit or less, the effect of the present invention can be even more effectively exhibited. The lower limit of the water swelling ratio of the resin film is not particularly limited. The resin film may have a water swelling ratio of 0.5% or more.

The water swelling ratio is measured as follows.

The resin film is cut out to obtain a resin film (test piece) with 1 cm length×3 cm width×100 μm thickness. The scaffold material may be formed into a film to obtain a test piece having the above size. The obtained test piece is immersed in 50 g of pure water at 37° C. for 24 hours, and the immersed test piece is filtered using a plain weave wire mesh with an opening of 200 mesh to obtain a swollen test piece. The water swelling ratio is calculated according to the following formula.

Water swelling ratio (%)=(Weight of swollen test piece (g)−Weight of test piece (g))/(Weight of test piece (g))×100

From the viewpoint of even more enhancing adhesiveness and proliferation of cells, the resin film preferably has a phase-separated structure. The phase-separated structure has at least a first phase and a second phase.

Examples of the phase-separated structure include microphase-separated structures such as a sea-island structure, a cylinder structure, a gyroid structure, and a lamellar structure. In the sea-island structure, for example, the first phase can be a sea portion and the second phase can be an island portion. In the cylinder structure, gyroid structure, or lamellar structure, for example, a phase having a largest surface area can be the first phase, and a phase having a second largest surface area can be the second phase. The resin film has a continuous phase and a discontinuous phase, thereby enhancing affinity with cells, and the adhesiveness and proliferation of cells can be even more enhanced.

The phase-separated structure is preferably a sea-island structure. The resin film preferably has a sea-island structure. In this case, the adhesiveness and proliferation of cells can be even more enhanced.

When the resin film has a sea-island structure, surface area fraction of the island portion (second phase) with respect to the entire surface of the resin film is preferably 0.01 or more, more preferably 0.1 or more, further preferably 0.2 or more, preferably 0.95 or less, more preferably 0.9 or less, and further preferably 0.8 or less. When the surface area fraction is the above lower limit or more and the above upper limit or less, the adhesiveness of cells can be even more enhanced.

When the resin film has a sea-island structure, it is preferable that the island portion contains a peptide portion. That is, it is preferable that the resin film has a sea portion and an island portion, and the island portion contains a peptide portion. In this case, adhesion domains of the cells are accumulated in the island portion, whereby the adhesiveness of cells can be further enhanced.

The presence or absence of a phase-separated structure can be confirmed by, for example, an atomic force microscope (AFM), a transmission electron microscope (TEM), a scanning electron microscope (SEM), or the like. Further, the surface area fraction can be obtained from a microscope observation image using image analysis software such as ImageJ.

The phase-separated structure can be formed, for example, by increasing the content of the peptide portion and forming a phase-separated structure between or within molecules of a peptide-conjugated acrylic resin.

(Cell Culture Vessel)

The cell culture vessel includes the above-mentioned resin film in at least a part of cell culture area. It is preferable that the cell culture vessel includes a vessel body and the resin film, and the resin film is arranged on the surface of the vessel body.

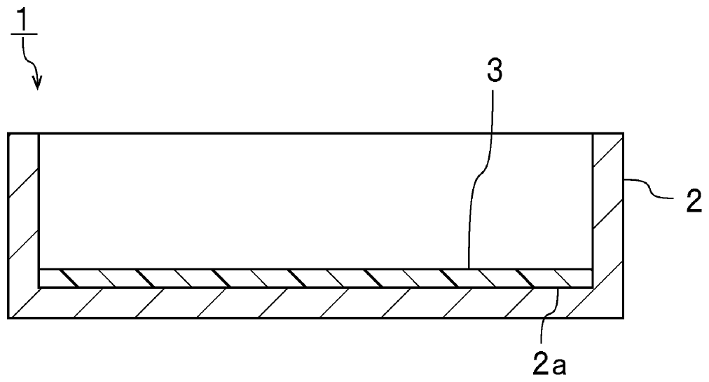

FIG. 1 is a cross-sectional view schematically showing a cell culture vessel according to an embodiment of the present invention.

A cell culture vessel 1 includes a vessel body 2 and a resin film 3. The resin film 3 is arranged on a surface 2a of the vessel body 2. The resin film 3 is arranged on the bottom surface of the vessel body 2. Cells can be cultured in plane by adding a liquid medium to the cell culture vessel 1 and seeding cells such as cell mass on the surface of the resin film 3.

The vessel body may include a first vessel body, and a second vessel body such as a cover glass on the bottom surface of the first vessel body. The first vessel body and the second vessel body may be separable. In this case, the resin film may be arranged on the surface of the second vessel body.

As the vessel body, a conventionally known vessel body (vessel) can be used. The shape and size of the vessel body are not particularly limited.

Examples of the vessel body include a cell culture plate provided with one or a plurality of wells (holes), a cell culture flask, and the like. The number of wells in the plate is not particularly limited. The number of wells is not particularly limited, and examples thereof include 2, 4, 6, 12, 24, 48, 96, 384, and the like. The shape of the well is not particularly limited, and examples thereof include a perfect circle, an ellipse, a triangle, a square, a rectangle, a pentagon, and the like. The shape of the bottom surface of the well is not particularly limited, and examples thereof include a flat bottom, a round bottom, unevenness, and the like.

The material of the vessel body is not particularly limited, and examples thereof include resins, metals, and inorganic materials. Examples of the resin include polystyrene, polyethylene, polypropylene, polycarbonate, polyester, polyisoprene, cycloolefin polymer, polyimide, polyamide, polyamideimide, (meth)acrylic resin, epoxy resin, silicone, and the like. Examples of the metal include stainless steel, copper, iron, nickel, aluminum, titanium, gold, silver, platinum, and the like. Examples of the inorganic material include silicon oxide (glass), aluminum oxide, titanium oxide, zirconium oxide, iron oxide, silicon nitride, and the like.

(Method for Culturing Cells)

Cells can be cultured using the scaffold material and the resin film. The method for culturing cells is a method for culturing cells using the scaffold material. The method for culturing cells is preferably a method for culturing cells using the resin film. Examples of the cells include the above-mentioned cells.

The method for culturing cells preferably includes a step of seeding cells on the scaffold material. The method for culturing cells preferably includes a step of seeding cells on the resin film. The cells may be a cell mass. The cell mass can be obtained by adding a cell detachment agent to a confluent culture vessel and uniformly crushing it by pipetting. The cell detachment agent is not particularly limited, but an ethylenediamine/phosphate buffer solution is preferable. The size of the cell mass is preferably 50 μm to 200 μm.

The present invention will be described in more detail below with reference to Examples and Comparative Examples. The present invention is not limited to these examples.

The content of structural units in the obtained peptide-conjugated acrylic resin was measured by 1H-NMR (nuclear magnetic resonance spectrum) after dissolving a synthetic resin in DMSO-d6 (dimethylsulfoxide). Also, the content of peptide in the peptide-conjugated acrylic resin was measured by FT-IR or LC-MS.

### Example 1

Preparation of Peptide-Conjugated Acrylic Resin:

Twenty-nine parts by weight of butyl acrylate, 3 parts by weight of 2-hydroxyethyl acrylate, and 1 part by weight of acrylic acid were dissolved in 30 parts by weight of tetrahydrofuran to obtain an acrylic monomer solution. One part by weight of Irgacure 184 (manufactured by BASF SE) was dissolved in the obtained acrylic monomer solution, and the obtained solution was applied onto a PET film. An acrylic resin solution was obtained by irradiating the coated material with light with a wavelength of 365 nm at an integrated light quantity of 2000 mJ/cm² using a UV conveyor device (“ECS301G1” manufactured by Eye Graphics Co., Ltd.) at 25° C. The obtained acrylic resin solution was vacuum dried at 80° C. for 3 hours to obtain an acrylic resin.

The obtained acrylic resin had a weight average molecular weight of about 100,000.

The obtained acrylic resin was dissolved in a butanol solution so as to have 3% by weight to obtain an acrylic resin-containing butanol solution. The obtained acrylic resin-containing butanol solution (150 μL) was discharged onto a ϕ22 mm cover glass (manufactured by Matsunami Glass Ind., Ltd., using 22 round No. 1 after removing dust with an air duster), and rotated at 2000 rpm for 20 seconds using a spin coater to obtain a smooth resin film.

Next, a linear peptide having an amino acid sequence of Gly-Arg-Gly-Asp-Ser (five amino acid residues, described as GRGDS in the table) was prepared. One part by weight of this peptide and 1 part by weight of 1-ethyl-3-(3-dimethylaminopropyl)carbodiimide hydrochloride (condensing agent) were added to phosphate buffered saline containing neither calcium nor magnesium so that the final concentration of the peptide is 1 mM to prepare a peptide-containing solution. One part by weight of this peptide-containing solution was added to the obtained resin film, and a carboxyl group of the acrylic resin and an amino group of Arg of the peptide were dehydrated and condensed. In this way, the peptide-conjugated acrylic resin (resin film) of Example 1 was obtained.

Preparation of Cell Culture Vessel:

A laminate of the obtained peptide-conjugated acrylic resin (resin film) and the cover glass was arranged on a polystyrene dish with a diameter of 22 mm to obtain a cell culture vessel.

### Examples 2, 3

Peptide-conjugated acrylic resins (resin films) having the configurations shown in Table 1 were each obtained in the same manner as in Example 1 except that the blending amounts of butyl acrylate and 2-hydroxyethyl acrylate were changed. In addition, cell culture vessels were each obtained in the same manner as in Example 1 except that each of the obtained peptide-conjugated acrylic resins were used.

### Comparative Example 1

A peptide-conjugated acrylic resin (resin film) having the configuration shown in Table 2 was obtained in the same manner as in Example 1 except that methoxy-triethylene glycol acrylate was used instead of butyl acrylate and 2-hydroxyethyl acrylate. In addition, a cell culture vessel was obtained in the same manner as in Example 1 except that the obtained peptide-conjugated acrylic resin was used.

### Comparative Example 2

A peptide-conjugated acrylic resin (resin film) having the configuration shown in Table 2 was obtained in the same manner as in Example 1 except that 2-hydroxyethyl acrylate was not used and the blending amount of butyl acrylate was changed. In addition, a cell culture vessel was obtained in the same manner as in Example 1 except that the obtained peptide-conjugated acrylic resin was used.

### Comparative Example 3

An acrylic resin (resin film) having the configuration shown in Table 2 was obtained in the same manner as in Example 1 except that the blending amounts of butyl acrylate and 2-hydroxyethyl acrylate were changed and a peptide and acrylic acid were not used. In addition, a cell culture vessel was obtained in the same manner as in Example 1 except that the obtained acrylic resin was used.

(Evaluation)

(1) Solubility Parameter (SP Value) of First Structural Part of Peptide-Conjugated Acrylic Resin

The solubility parameter (SP value) calculated by Okitsu's equation of a first structural part of the peptide-conjugated acrylic resin was calculated.

(2) Compressive Elastic Modulus of Resin Film in Ion Exchange Water

A cell culture vessel having a resin film formed of a cell culture scaffold material was put in a beaker filled with ion exchange water, and then the beaker was left in a constant temperature bath at 37° C. for 24 hours. The cell culture vessel immersed for 24 hours was taken out with the resin film immersed in ion exchange water. The compressive elastic modulus of the resin film was measured in ion exchange water at a frequency of 1 Hz in accordance with ISO14577-1 using a nanoindenter device (Triboindenter, manufactured by Hysitron Inc.). The compressive elastic modulus was calculated according to the following formula.

Compressive elastic modulus=√π×(Slope of load-displacement curve in elastic region)/(2×√(Contact projected area))

The elastic region refers to a region where the slope of the load-displacement curve is constant. Also, the contact projected area refers to the area where an indenter and the sample come into contact with each other.

Berkovich (triangular pyramid shape, tip diameter R is several hundred nanometers) was used as the indenter, and the indentation depth was set to 50 nm.

(3) Water Swelling Ratio of Resin Film

The obtained resin film was cut out to obtain a resin film (test piece) with 1 cm length×3 cm width×100 μm thickness. The obtained test piece was immersed in 50 g of pure water at 37° C. for 24 hours, and the immersed test piece was filtered using a plain weave wire mesh with an opening of 200 mesh to obtain a swollen test piece. The water swelling ratio was calculated according to the following formula.

Water swelling ratio (%)=(Weight of swollen test piece (g)−Weight of test piece (g))/(Weight of test piece (g))×100

(4) Presence or Absence of Sea-Island Structure

The obtained resin film was immersed in PBS solution for 30 minutes. The immersed resin film was observed with an atomic force microscope (AFM, “Dimension XR” manufactured by Bruker). Under measurement conditions where peak set point was set to 2 nN in QNM mode, the range of 1 μm×1 μm was observed. The presence or absence of the sea-island structure was determined by comparing the obtained height mapping image and elastic modulus mapping image.

(5) Culture Evaluation of Cells (Adhesiveness of Cells)

The following liquid medium and ROCK (Rho-associated kinase)-specific inhibitor were prepared.

TeSR E8 medium (manufactured by STEMCELL Technologies Inc.)

ROCK-Inhibitor (Y27632)

Phosphate buffered saline (1 mL) was added to the obtained cell culture vessel, and the mixture was allowed to stand in an incubator at 37° C. for 1 hour, then the phosphate buffered saline was removed from the cell culture vessel.

Colonies of h-iPS cells 253G1 in a confluent state were placed in a dish of ϕ35 mm, 1 mL of a 0.5 mM ethylenediamine/phosphate buffer solution was added thereto, and the mixture was allowed to stand at room temperature for 2 minutes. The ethylenediamine/phosphate buffer solution was removed, followed by pipetting with 1 mL of TeSR E8 medium to obtain a cell mass crushed to a size of 50 μm to 200 μm. The obtained cell mass (cell number 0.2×10⁵ cells) was clamp-seeded on the resin film of the cell culture vessel.

At the time of seeding, 1.5 mL of liquid medium, and a ROCK-specific inhibitor in an amount so as to have a final concentration of 10 μM were added to the cell culture vessel, and the cells were cultured in an incubator at 37° C. and a CO₂ concentration of 5%. Thereafter, the cells were cultured for 5 days by repeating an operation of exchanging the liquid in the cell culture vessel with 1.5 mL of fresh liquid medium every 24 hours. At that time, the cells free or suspended from the resin film were collected and discarded by pipetting.

The entire resin film after culturing for 5 days was photographed with a phase-contrast microscope, and occupied area of the cell mass relative to the area of the resin film was calculated. The adhesiveness of cells was determined according to the following criteria.

<Criteria for Culture Evaluation of Cells (Adhesiveness of Cells)>
AA: 70% or more of the occupied area of the cell mass
A: 50% or more and less than 70% of the occupied area of the cell mass
B: Less than 50% of the occupied area of the cell mass

Details and results are shown in Tables 1 and 2 below.

TABLE 1

| | | | | | Example | Example | Example |
|---|---|---|---|---|---|---|---|
| | | | | | 1 | 2 | 3 |
| Peptide- | First structural | (Meth)acrylic | Butyl acrylate | mol % | 80 | 55 | 35 |
| conjugated | part having no | acid ester | 2-Hydroxyethyl acrylate | mol % | 10 | 35 | 55 |
| acrylic | peptide portion in | | Methoxy-triethylene | mol % | — | — | — |
| resin | side chain | | glycol acrylate | | | | |
| | | Solubility parameter (SP value) | | (cal/cm³)^{1/2} | 9.8 | 10.2 | 10.5 |
| | Second structural | Compound X | Acrylic acid | mol % | 5 | 5 | 5 |
| | part having | Peptide | GRGDS | mol % | 5 | 5 | 5 |
| | peptide portion | | | | | | |
| | in side chain | | | | | | |

| | | | | | |
|---|---|---|---|---|---|
| Resin film | Compressive elastic modulus in ion exchange water | GPa | 2.3 | 2.8 | 3.6 |
| | Water swelling ratio | % | 15 | 34 | 49 |
| | Presence or absence of sea-island structure | | Present | Present | Present |

| | | | |
|---|---|---|---|
| Culture evaluation of cells (adhesiveness of cells) | A | AA | A |

TABLE 2

| | | | | | Comparative | Comparative | Comparative |
|---|---|---|---|---|---|---|---|
| | | | | | Example | Example | Example |
| | | | | | 1 | 2 | 3 |
| Peptide- | First structural | (Meth)acrylic | Butyl acrylate | mol % | — | 90 | 61 |
| conjugated | part having no | acid ester | 2-Hydroxyethyl acrylate | mol % | — | — | 39 |
| acrylic | peptide portion in | | Methoxy-triethylene | mol % | 90 | — | — |
| resin | side chain | | glycol acrylate | | | | |
| | | Solubility parameter (SP value) | | (cal/cm³)^{1/2} | 10.8 | 9.6 | 10.2 |
| | Second structural | Compound X | Acrylic acid | mol % | 5 | 5 | — |
| | part having | Peptide | GRGDS | mol % | 5 | 5 | — |
| | peptide portion | | | | | | |
| | in side chain | | | | | | |

| | | | | | |
|---|---|---|---|---|---|
| Resin film | Compressive elastic modulus in ion exchange water | GPa | 0.8 | 1.7 | 2.5 |
| | Water swelling ratio | % | 91 | 11 | 28 |
| | Presence or absence of sea-island structure | | Present | Present | Absent |

| | | | |
|---|---|---|---|
| Culture evaluation of cells (adhesiveness of cells) | B | B | B |

What is claimed is:

1. A cell culture scaffold material comprising a peptide-conjugated acrylic resin, wherein
the peptide-conjugated acrylic resin has a first structural part having no peptide portion in a side chain and a second structural part having a peptide portion in a side chain, and
solubility parameter calculated by Okitsu's equation for the first structural part is 9.7 (cal/cm³)^{1/2} or more and 10.7 (cal/cm³)^{1/2} or less.

2. The cell culture scaffold material according to claim 1, wherein the first structural part has a poly(meth)acrylic acid ester skeleton.

3. The cell culture scaffold material according to claim 1, wherein the number of amino acid residues in the peptide portion in the second structural part is 10 or less.

4. A resin film formed of the cell culture scaffold material according to claim 1.

5. The resin film according to claim 4, wherein compressive elastic modulus at a frequency of 1 Hz measured in ion exchange water after immersing the resin film in the ion exchange water at 37° C. for 24 hours in accordance with ISO14577-1 using a nanoindenter device is 1 GPa or more.

6. The resin film according to claim 4, which has a water swelling ratio of 70% or less.

7. The resin film according to claim 4, which has a sea-island structure, wherein an island portion in the sea-island structure contains the peptide portion.

8. A cell culture vessel equipped with the resin film according to claim 4 in at least a part of cell culture area.

9. A method for culturing cells, comprising the step of seeding cells on the resin film according to claim 4.
